(19) United States
(12) Patent Application Publication
(10) Pub. No.: US 2017/0128278 A1
(43) Pub. Date: May 11, 2017

(54) DEVICE FOR FOLDING AT LEAST ONE WEB FEEDING ALONG A PLANT FOR PRODUCING ABSORBENT SANITARY ARTICLES

(71) Applicant: GDM S.p.A., Bologna (IT)

(72) Inventors: Matteo PIANTONI, Albino (Bergamo) (IT); Valerio SOLI, Bologna (IT)

(21) Appl. No.: 15/118,408

(22) PCT Filed: Mar. 12, 2015
(86) PCT No.: PCT/IB2015/051817

§ 371 Date: Aug. 11, 2016

(30) Foreign Application Priority Data
Mar. 18, 2014 (IT) BO2014A000138

(51) Int. Cl. A61F 13/15

(52) U.S. Cl. CPC A61F 13/15804; A61F 13/15747; A61F 13/15764; A61F 13/15682; A61F 13/15772; A61F 2013/15829; A61F 2013/15796

(57) ABSTRACT

A device for folding a web for producing absorbent sanitary articles along at least one longitudinal fold line parallel to its feed direction includes two diverting elements positioned on opposite sides of the web one after the other, to operate in succession on respective lateral portions of the web and allow transversal folding of one lateral portion on the other and both on the web. Each diverting element has a diverting surface which is used for folding and which is equipped with a plurality of holes to emit jets of air under pressure at a tangent to the diverting surface, directed transversely to the feed direction of the web along the same direction as the direction along which the two lateral portions are transversely folded by the diverting surface and defining between the latter and the web a laminar fluid bed.

### TECHNICAL FIELD

This invention relates to a device for folding at least one web feeding along a plant for producing absorbent sanitary articles.

More specifically, this invention relates to a folding device which can be used for folding at least one web comprising a succession of disposable nappies during production, mutually interconnected in pairs.

### BACKGROUND ART

During the production of nappies it is normal practice to longitudinally feed a web, or two or more mutually superposed webs, with continuous motion and perform processes consisting, for example, in operations for removing portions of sheet material, or applying accessory elements such as, for example, elastics, closing tabs etc.

In this description reference is made, for simplicity, to the folding of a single web.

It is common practice, in particular towards the end of the steps for processing the web and before separating the individual nappies, to transversely fold the web at both its sides.

This operation is usually performed by sliding the web in contact with specific fixed lateral guides with a spiral or helical profile, which progressively fold the sides of the web towards each other, normally superposing one on the other.

The regularity with which the fold is performed is certainly not satisfactory, in particular when the web has large transversal dimensions and moves forward at high speeds.

In effect, the friction which is created between the web and the lateral guides frequently leads to folding of the latter along fold lines which are not uniformly rectilinear and not parallel to the feed direction of the web as well as wrinkling and incorrect positioning of the lateral portions of the web.

Also, in the prior art apparatuses, the folding of the lateral sides of the web is performed in a substantially simultaneous manner, with consequent fast superposing of one of the sides on the other.

This all prevents, in these apparatuses, the provision of inspection devices capable of checking whether the folding of the side of the web is being performed in a regular fashion and whether the second side correctly superposes the first side following fold lines which are straight and correctly positioned.

Moreover, the fact of applying on the web accessory elements such as, for example, elastics, closing tabs and the like makes the lateral zones of the web heavier than the rest of the web. That means that during the step of folding the two edges the weight tends to cause folding to start too early, with consequent folding irregularities along the lines provided.

### DISCLOSURE OF THE INVENTION

The aim of this invention is to provide a device for folding at least one web feeding along a plant for producing absorbent sanitary articles which overcomes the above-mentioned drawbacks.

This aim is achieved by a device for folding at least one web having the features set out in one or more of the appended claims and, more specifically, which comprises feeding means for feeding a continuous web along a feed direction which is oriented parallel with the web, and at least one diverting element having a diverting surface for folding the web.

The device is configured to fold the web along at least one longitudinal fold line parallel to a feed direction of the continuous web in order to start transversely folding at least one lateral portion of the web onto a central portion of the web.

According to one aspect of the invention, the diverting surface of the diverting element has a plurality of holes designed to emit respective jets of air under pressure at a tangent to the diverting surface and defining between the latter and the web a laminar fluid bed.

The invention accordingly provides a device for folding at least one web feeding along a plant for producing absorbent sanitary articles, as described in one or more of the appended claims.

### BRIEF DESCRIPTION OF THE DRAWINGS

The invention is described below with reference to the accompanying drawings, which illustrate two preferred non-limiting embodiments of it, and in which:

FIGS. 1 and 2 schematically show, respectively, a front view and a plan view of a portion of a plant for producing absorbent sanitary articles equipped with the folding device made in accordance with this invention;

FIG. 3 is a schematic perspective view of a portion of the folding device of FIGS. 1 and 2;

FIGS. 4 and 5 schematically show in cross section respective variant embodiments of some details of the folding device of FIGS. 1 to 3;

FIG. 6 is a perspective view of a web obtained after two transversal folding steps performed by the apparatus of FIGS. 1 to 5;

FIGS. 7 and 8 show two different absorbent sanitary articles obtained by transversely cutting the web of FIG. 6;

FIG. 9 is a schematic plan view of a further embodiment of the folding device of FIG. 2.

### DETAILED DESCRIPTION OF PREFERRED EMBODIMENTS OF THE INVENTION

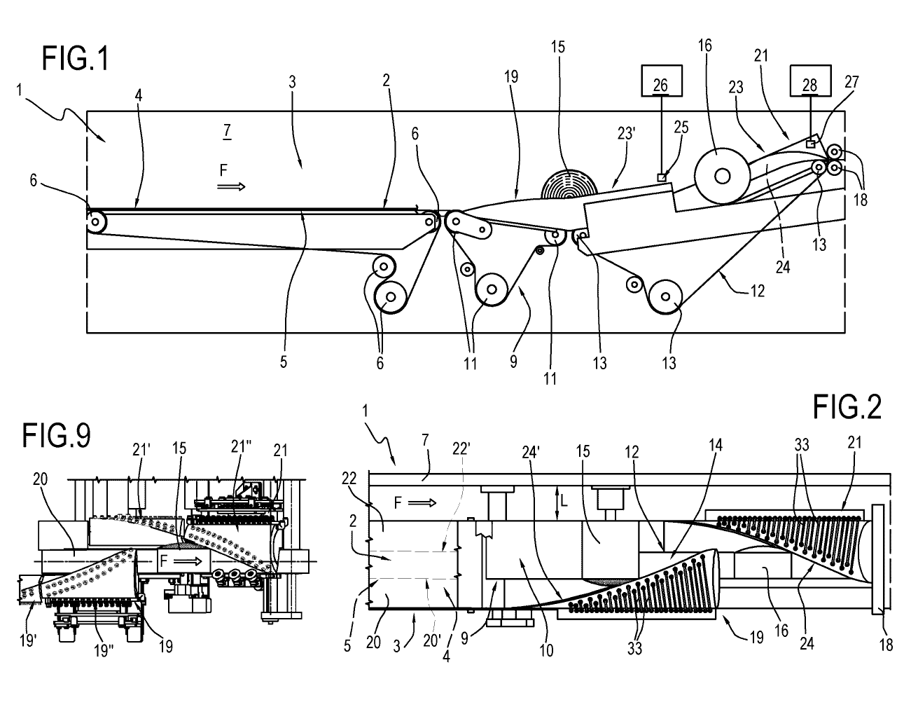

As illustrated in FIGS. 1 and 2, the numeral 1 denotes in its entirety a folding device capable of transversely folding at least one continuous web 2 feeding with longitudinal motion.

Hereinafter, even where not specified, the expression “web 2” is used to refer to a continuous web of material.

More specifically, as will become clearer as this description continues, the web 2 is designed to be folded along two longitudinal fold lines, labelled, respectively, 20′ and 22′.

The two fold lines define two lateral portions of the web, labelled, respectively, 20 and 22, the first of which designed to be folded on the web 2 and the second designed to be folded both on the web and on the portion 20 folded previously.

The device 1 is installed in a plant which produces absorbent sanitary articles and respective portions of which are schematically illustrated in FIGS. 1 and 2 and it is capable of processing a web, or several mutually superposed and, normally, mutually connected webs, but in this description, for simplicity, reference will be made to the processing of a single web.

The web 2, coming from operating stations of the plant not illustrated, is fed with continuous motion by specific feeding means 3 configured to move it along its direction of extension.

Preferably, the web 2 initially lies in a horizontal plane above belt conveying means which extend substantially horizontally and which are denoted in their entirety by the reference numeral 3.

The belt conveying means 3 comprise a first conveyor belt 4, equipped with an upper transportation branch 5 positioned horizontally and associated, in a known manner, to a suction source, not illustrated, to keep the web 2 in close contact with the branch 5.

The conveyor belt 4 is looped around a plurality of pulleys 6 rotatable about respective horizontal axes parallel to each other, one of which is motor-driven. The shafts which coaxially support the pulleys 6 are supported by a vertical wall 7 perpendicular to them and forming part of a base for supporting the folding device 1 which extends parallel to a horizontal extension and feed direction F of the upper transportation branch 5, which coincides substantially with the feed direction of the web 2.

The belt conveying means 3 comprise a second conveyor belt 9, having an infeed end next to an outfeed end of the first conveyor belt 4 and equipped with an upper transportation branch 10 inclined slightly downwards with respect to the horizontal starting from the infeed end of it next to the conveyor belt 4.

The conveyor belt 9 is looped around a plurality of pulleys 11 rotatable about respective horizontal axes parallel to each other, one of which is motor-driven. The shafts which coaxially support the pulleys 11 are also supported by the above-mentioned wall 7, which is perpendicular to them.

As shown in FIG. 2, the conveyor belt 9 has a width less than that of the conveyor belt 4, and has a respective lateral edge lying in a vertical plane of the lateral edge of the conveyor belt 4 located close to the wall 7.

The belt conveying means 3 also comprise a third conveyor belt 12, having an infeed end next to an outfeed end of the second conveyor belt 9 and equipped with an upper transportation branch 14 inclined slightly upwards with respect to the horizontal starting from the infeed end of it next to the conveyor belt 9. The conveyor belt 12 is looped around a plurality of pulleys 13 rotatable about respective horizontal axes parallel to each other, one of which is motor-driven. The shafts which coaxially support the pulleys 13 are also supported by the above-mentioned wall 7, which is perpendicular to them.

The upper transportation branch 10 of the conveyor belt 9 and the upper transportation branch 14 of the conveyor belt 12 can, if required, be associated in a known manner, if necessary only partly, with suction means not illustrated so as to keep the web 2 in close contact with their transportation surfaces.

As shown in FIG. 2, the conveyor belt 12 has a width less than that of the conveyor belt 9, and has a respective lateral edge lying in a vertical plane of the lateral edge of the conveyor belt 9 in alignment with this, which is positioned, unlike the previous lateral edge, away from the wall 7. As shown in particular in FIG. 2, an outfeed portion of the conveyor belt 9 and an infeed portion of the conveyor belt 12 are superposed by contact means.

The contact means comprise a roller 15, rotatable about a horizontal axis and perpendicular to the wall 7 and having its cylindrical peripheral surface positioned close to the surface of the transportation branches 10 and 14 of the conveyor belts 9 and 12. In the same way, a middle portion of the conveyor belt 12 is superposed by a contact means comprising a roller 16, rotatable about a horizontal axis and perpendicular to the wall 7 and having its cylindrical peripheral surface positioned close to the transportation branch 14 of the conveyor belt 12.

The free circular edges of the two contact rollers 15 and 16 are aligned with the longitudinal fold lines 20′ and 22′ of the web 2.

It is thus evident that the above-mentioned circular edges allow a contact element to be made for folding at the two predetermined longitudinal fold lines 20′ and 22′.

The contact being obtained through the two rollers 15 and 16 guarantees a continuous and progressive contact which acts on the two longitudinal fold lines 20′ and 22′.

At an outfeed end of the third conveyor belt 12 there are two counter-rotating rollers 18 in substantially mutual contact with each other, rotatable about respective horizontal axes perpendicular to the wall 7 which define means of pulling and feeding the web 2 sliding between the two rollers 18.

At an infeed portion of the conveyor belt 9 there is, immediately above the level of the transportation branch 10, a first diverting element 19 designed to act on a lateral portion 20 of the web 2 to fold it transversely above the remaining part of the web 2, that is to say, above a central portion of it. More precisely, the first diverting element 19 is designed to act on the lateral portion 20 of the web 2 which is furthest from the wall 7.

Similarly, at a substantially middle zone of the conveyor belt 12 there is, immediately above the level of the transportation branch 14, a second diverting element 21 designed to act on a lateral portion 22 of the web 2 positioned close to the wall 7 to fold it transversely above the remaining part of the web 2.

It should be noted that the arrangement of the upper transportation branches 10 and 14 of the conveyors 9 and 12 is chosen in such a way as to reduce the tension of the web 2 in the transit zone in contact with the upper transportation branches 10 and 14, and this situation facilitates the above-mentioned folding of the web 2 under the action of the diverting elements 19 and 21.

The two diverting elements 19 and 21 are arranged in succession with reference to the feed direction of the web 2, and they are structured in the same way, except for the fact that their purpose is to fold different respective lateral portions 20, 22 of the web 2.

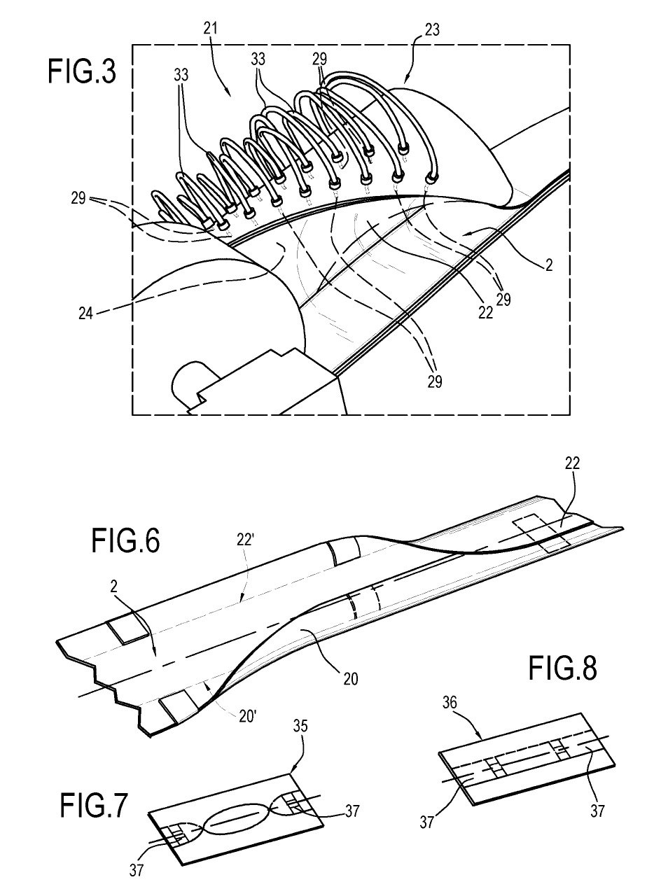

Only the structure of the second diverting element 21 is therefore described below, shown in detail in FIG. 3, and the component portions of the first diverting element 19 will be assigned reference numerals equal to those of the component portions of the diverting element 21, but marked with a superscript.

The diverting element 21 basically comprises an elongate body 23 equipped with a diverting surface 24 for folding the web 2, along which the above-mentioned lateral portion 22 of the web 2 closest to the wall 7 slides in the manner which will be described in more detail below.

The diverting surface 24 has a spiral surface profile which extends in the feed direction F of the web 2 for acting uninterruptedly and progressively on the lateral portion 22.

The diverting surface 24 may have in its most extreme shape a helical profile.

In other words, the diverting surface 24 intercepts the above-mentioned portion 22 of the web 2 firstly below in such a way as to fold it gradually upwards and away from the wall 7, and then fold it above the portion of the remaining part of the web 2 along a longitudinal fold line parallel to the direction F.

During the step of folding the lateral portion 20 of the web 2, the first diverting element 19 is designed to act on the web 2 in a manner similar to that just described with reference to the diverting element 21, to engage with its diverting surface 24′ the lateral portion 20 of the web 2 and fold it transversely above the remaining portion of the web 2 along a longitudinal fold line parallel to the direction F. Simultaneously, the contact roller 15 continuously abuts the longitudinal fold line 20′ and allows the portion 20 to fold in a precise manner along the line 20′.

The web 2 is engaged, in the manner described above, by the diverting element 21, and its lateral portion 22 is folded, at least partly, above the lateral portion 20 folded previously.

Also in this subsequent folding step, the contact roller 16, in a similar manner to that described for the roller 15, continuously abuts the longitudinal fold line 22′ and allows the portion 22 to fold precisely along the line 22′ and position itself correctly both above the portion 20 and above the web 2.

As illustrated in FIGS. 2, 3 and 6, as the web 2 in contact with the diverting surfaces 24 and 24′ feeds along the direction F, the above-mentioned folds are formed and completed and the fully folded web 2 rests on the upper transportation branch 14 of the conveyor belt 12, of which the folded web 2 substantially equals the width.

The web 2 folded in a manner described, along a first and then along a second rectilinear fold line, passes between the two above-mentioned counter-rotating rollers 18, and is then fed towards further processing stations, not illustrated.

Preferably, an inspection device 25 for checking the shape of the web 2 which has undergone the first folding by the diverting element 19 can be positioned immediately downstream of the diverting element 19 with reference to the direction F (FIG. 1), and the electrical signals indicating the measurements of the inspection device 25 can be sent to a processing unit, of a known type, schematically illustrated in the form of a block 26. A similar inspection device 27, with an associated processing unit 28, can also be located downstream of the diverting element 21 with reference to the direction F, to check the regularity of shape of the fully folded web 2.

As shown in FIGS. 3 to 5, the diverting surface 24, 24′ of the diverting element 19, 21 has a plurality of holes 29, which are connected to a source 30 of compressed air.

The holes 29 define small amplifiers of air and constitute nozzles which are able to generate jets of air under pressure.

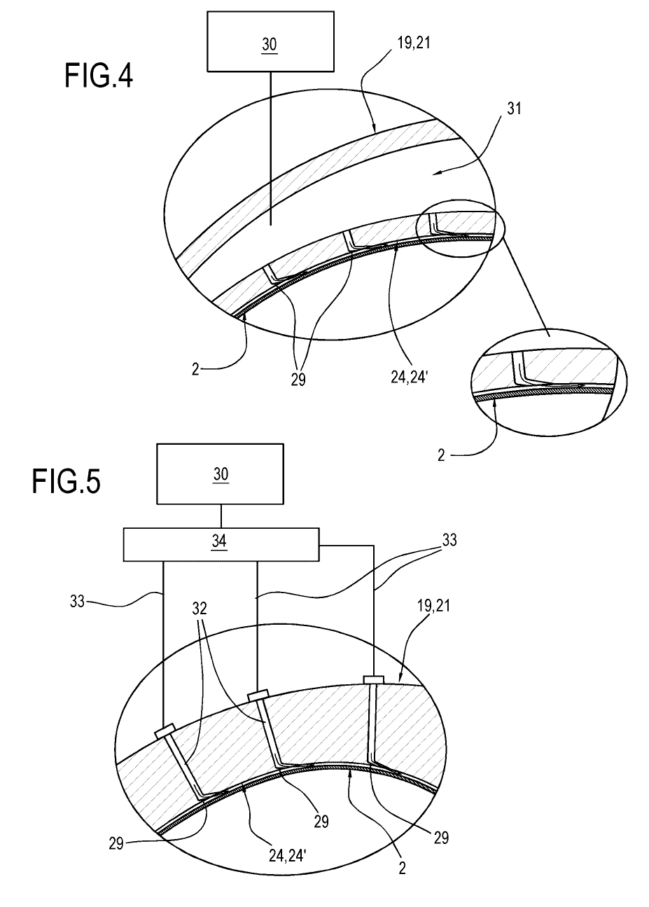

More specifically, in a first embodiment of the diverting element 19, 21 illustrated in FIG. 4, the diverting element 19, 21 is provided with a cavity 31, which communicates with all the holes 29 present on the diverting surface 24, 24′ which delimits the portion of the diverting element 19, 21 designed to enter into contact with the web 2.

The cavity 31 is in direct communication with the above-mentioned source 30 of compressed air.

In a second embodiment of the diverting element 19, 21 illustrated in FIG. 5, the diverting element 19, 21 is made solid, that is, without the inner cavity, and is crossed by a plurality of channels 32, each of which has one end coinciding with one of the above-mentioned holes 29 and the other end connected, by a tubular element 33, to an internally hollow manifold element 34 which communicates with the above-mentioned source 30 of compressed air.

The end zones of the holes 29 of each diverting surface 24, 24′ extend according to respective directions tangential to the diverting surface 24, 24′.

The plurality of jets of pressurized air coming from the holes 29 define, between the above-mentioned diverting surface 24, 24′ and the web 2 which travels below it, a laminar fluid bed.

According to a preferred embodiment, the above-mentioned end zones of the holes 29 of each diverting surface 24, 24′ extend according to respective directions transversal to the feed direction F of the web 2.

The shape of the diverting element 19, 21 described and of the respective holes 29 and the consequent generation of the laminar fluid bed interposed between the diverting surface and the web 2, allows the web 2 to slide with extremely low friction near the diverting surface 24, 24′.

The air emitted through the holes 29, in an extremely limited quantity and with a very low over-pressure relative to atmospheric pressure, due to the Coanda effect and in accordance with Bernoulli's principle, tends to attract the web 2 to the diverting surface 24, 24′ during transversal folding. This attraction results from the fact that the thin layer of air coming from the holes 29 defining the above-mentioned laminar fluid bed present between the web 2 and the diverting surface 24, 24′, as a result of the relatively high speed with which it transits in contact with the web 2, adopts a pressure less than the atmospheric pressure to which the surface of the web 2 is subjected. In other words, the plurality of holes 29 and the relative jets of air injected tangentially on the diverting surface 24, 24′ produce a laminar flow of air along the entire surface in question using the above-mentioned Coanda effect which involves a large volume of air of the area surrounding the diverting surface, together with a relative small quantity of compressed air coming from the holes 29.

The regularity of folding of the lateral portions of the web 2 is guaranteed by this attraction exerted by the diverting surface 24, 24′; in effect, the portion of the web 2 that at each instant transits in contact with the blade of air of the diverting surface 24, 24′ cannot detach itself due to gravity and fall downwards.

Consequently, the two portions 20 and 22 of the web provided with the above-mentioned additional elements and therefore heavier than the rest of the web 2 do not fall in an irregular manner onto the web 2 but are held in the correct position during their folding, due to the attraction effect.

Moreover, the fact that the folding of the lateral portions 20, 22 of the web 2 is not performed by the diverting elements 19 and 21 in a substantially simultaneous manner makes it possible to use the inspection device 25 with associated processing unit 26 to check the regularity of the folds. Lastly, it should be noted that, in the embodiment illustrated, solely by way of an example, the above-mentioned holes 29 are made only in the outer zone of the diverting surfaces 24, 24′, that is to say, close to the free edge of the diverting surfaces, which the holes reach when the portions 20, 22 of the web 2 provided with the above-mentioned accessory elements are being folded.

In this embodiment, the effect of attraction which is generated on account of the laminar fluid bed according to the methods and with reference to the physical principles of Coanda and Bernoulli described above is limited to only the heaviest zone of the two portions 20 and 22.

Consequently, during the folding step, the innermost parts of the two lateral portions 20 and 22 of the web 2 slide in contact with the respective diverting surfaces 24, 24′.

The web 2 folded in the manner described above is then cut transversely in a cyclic fashion to produce partly folded absorbent sanitary articles.

FIGS. 7 and 8 show, respectively, two absorbent sanitary articles 35 and 36 of known type folded transversely, having different shapes which can be obtained using the above-mentioned operations for folding the web 2.

These absorbent sanitary articles 35 and 36 are associated with connecting elements 37 of known type, consisting, for example, of adhesive strips, which are applied, in a manner not illustrated, to the web 2 before its arrival close to the folding device 1.

Obviously, it would be possible to provide only one of above-mentioned diverting elements 19 and 21, to fold only one of the above-mentioned lateral portions 20, 22 of the web 2, it being understood that what characterizes the folding device 1 itself and makes it particularly advantageous is the fact that the diverting surface 24, 24′ is provided with the above-mentioned holes 29 designed to emit air at a pressure greater than atmospheric pressure.

Lastly, as illustrated in FIG. 9, each diverting element 19, 21 is divided into at least two separate portions, labelled 19′, 19″ and 21′ and 21″, and positioned one after another in such a way as to maintain the continuity of the respective diverting surfaces 24, 24′ for folding the web (2).

1. A device for folding at least one continuous web feeding along a plant for producing absorbent sanitary articles, the continuous web being designed to be folded along at least one longitudinal fold line parallel to a feed direction of the continuous web to transversely fold at least one lateral portion thereof onto a central portion of the web itself; the device comprising feeding means for feeding a continuous web along a feed direction which is oriented parallel with the web, and at least one diverting element having a diverting surface for folding the web; wherein
the diverting surface of the diverting element has a plurality of holes designed to emit respective jets of air under pressure at a tangent to the diverting surface and defining between the latter and the web a laminar fluid bed.

2. The folding device according to claim 1, wherein the jets of air under pressure are directed transversely to the feed direction of the web and determine a laminar fluid bed directed transversely to the web.

3. The folding device according to claim 1, wherein the jets of air under pressure continue along the diverting surface following the same direction as the direction along which the at least one lateral portion is transversely folded onto the web by the selfsame diverting surface.

4. The folding device according to claim 1, wherein the diverting surface has a helical surface profile which extends in the feed direction of the web so as to act uninterruptedly and progressively on the at least one lateral portion, in such a way as to fold it towards the remaining part of the web.

5. The folding device according to claim 1, wherein it comprises at least two diverting elements designed to operate on respective lateral portions of the web.

6. The folding device according to claim 1, wherein at least two diverting elements are positioned on opposite sides of the web and one after the other with reference to the feed direction of the web.

7. The folding device according to claim 1, wherein each diverting element divided into at least two separate portions, arranged in succession in such a way as to maintain the continuity of the respective diverting surfaces for folding the web.

8. The folding device according to claim 1, wherein each diverting element is provided with a cavity, which communicates with the holes of the diverting surface; the cavity being in communication with a source of compressed air.

9. The folding device according to claim 1, wherein each diverting element is crossed by a plurality of channels, each of which has one end coinciding with one of the holes of the respective diverting surface and the other end connected by a tubular element to an internally hollow manifold element associated with the source of compressed air.

10. The folding device according to claim 1, wherein the end zones of the holes of each diverting surface extend according to respective directions substantially tangential to the diverting surface and transversal to the feed direction of the web.

11. The folding device according to claim 1, wherein the holes are only provided in the outer zone of the diverting surfaces, that is to say, close to their free edge.

12. The folding device according to claim 1, wherein it comprises an inspection device which is used to check the conformity of the transversal folding of the web and which is positioned in a zone downstream of at least one diverting element with reference to the feed direction of the web; a unit being provided for processing the electrical signals indicating the measurements taken by the inspection device.

13. The folding device according to claim 1, where the web, during its transversal folding by each of the diverting elements, is carried by conveyor means, wherein it comprises contact means designed to abut the longitudinal fold lines.
